(19) United States
(12) Patent Application Publication
(10) Pub. No.: US 2013/0309656 A1
(43) Pub. Date: Nov. 21, 2013

(54) Antibody detection method and device for a saliva sample from a non-human animal

(71) Applicant: David C. Davis, Citrus Heights, CA (US)

(72) Inventor: David C. Davis, Citrus Heights, CA (US)

(21) Appl. No.: 13/506,798

(22) Filed: May 17, 2012

(51) Int. Cl. G01N 21/75; C12M 1/34

(52) U.S. Cl. 435/5; 422/69; 436/501; 435/287.2

(57) ABSTRACT

A rapid test apparatus, system, and method of use utilizing lateral flow immunoassay (LFIA) detection of a selected ligand in a liquid sample from a body fluid such as saliva in a pet in which antibodies and their complimentary antigens are used with detection-nanoparticles to provide a visual or measurable end point indicator in which the method measures the exposure to viruses in the canine from Canine Parvovirus (CPV) and/or Canine Distemper virus (CDV).

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

A rapid test apparatus, system, and method of use is disclosed for the lateral flow immunoassay (LFIA) detection of a selected ligand in a liquid sample from a body fluid such as saliva in a pet such as a dog. The test is rapid, ultrasensitive, and cost effective for routine use in the field for in vitro testing for recent exposure to infectious disease or modified live virus vaccine or long term antibody response. The disclosed test includes the use of antibodies and their complimentary antigen using nanoparticles such as a gold reporter to provide a visual or measurable end point indicator. Further and in particular, the subject invention refers to a method for measuring the exposure to viruses in the canine from Canine Parvovirus and/or Canine Distemper virus. Also, described is a cassette for use singly or side-by-side to measure a plurality of antibodies in the saliva of the dog.

2. Description of Related Art

The canine population, Canis familiaris, is susceptible to a wide range viral infections that are a source of significant morbidity and mortality. Two of these agents are the viruses Canine Parvovirus (CPV) and Canine Distemper (CDV). These diseases are considered to be two of the most serious/important viral diseases in dogs (Yang, et al., Appel, M. J., et al.).

At present, in the United States, most veterinarians administer a modified live virus vaccine containing antigenic components to CPV and CDV to puppies between 6 and 16 weeks of age in order to raise their antibody status to a protective level that will bind to viral particles and prevent the virus causing disease.

Diagnostic techniques for identification of antibodies against CPV infection includes enzyme-linked immunoassay (ELISA), serum viral neutralization (SVN), immune fluorescence assay (IFA), hemagglutination inhibition (HI)(Carmichael, et al.), enzyme immunoassay (EIA) microdot, and lateral flow immunoassay (LFIA).

CPV is a member of the family parvoviridae which includes parvoviruses that affect dogs, cats, raccoons, skunks, mink, humans, and seals. CPV mainly affects dogs, but it can also cause severe illness in wild cats such as lions, wolves, foxes, raccoons, and skunks.

CDV is, likewise, a serious disease. This virus is a member of the Paramyxoviridae, genus Morbillivirus. Diseases within this genus also includes measles, rinderpest, phocine (seal) distemper virus, peste-des-petits-ruminants virus, and cetacean morbillivirus. This virus is especially a problem in urban areas were unvaccinated animals are able to spread the virus. It is a problem with some animal control facilities where dogs are congregated together. Even though vaccination exists for dogs against CDV, the incidence of CDV infection has increased in both vaccinated and unvaccinated dogs (Appel, M. J., et al., 1979, Iwatsuki, K. et al., 1997).

The CDV virus is a member of the Morbillivirus of the family Paramyxoviridae (Zipperle, et al., 2010). The CDV viral particle is an enveloped non-segmented negative stranded RNA virus that injects its genetic material into the target cell by fusing with the host cell through hemagglutination (H) and then fusion proteins (F). The nucleocapsid protein (N) has recently been propagated in the baculovirus recombinant system. In this system the recombinant baculovirus-expressed CDV nucleocapsid protein (N) is produced with high efficiency in the larva of Heliothis virescens. Large amounts of N-protein of CDV can be grown for use in diagnostic testing for CDV infection (von Messling, V., 1999).

Vaccination exists for these two agents. However, vaccinations may not be effective in all patients with a significant number of dogs not maintaining protective titers at one year's time (McCaw, D. L., et al., 1998). Maternally-derived antibodies may interfere with a pup's ability to respond to puppy vaccinations because these maternal antibody levels may bind to the vaccine given, thereby negating its effect to protect the puppy against CPV and/or CDV. Thus, what was thought to be a protected pup can now come down with CDV and/or CPV. Pups can be nursed through CPV only to die of CDV, as an example.

Data from one vaccine company indicates that dogs are protected up to 3 years with their one year vaccines. However, examination of the data shows that the same dogs were not followed each time through the period. Most vaccine manufacturer's inserts/instructions still recommend an annual vaccine. Others have advocated that dogs may not have to be vaccinated at all once they have received their puppy shots and a one-year annual booster. Recommendations have been presented by various veterinary schools to recommend vaccination every 3 years after the first annual vaccination for CPV and CDV (Green and Levy, 2012). However, other studies by McCaw, et al., (1998), have found that 33 of 122 dogs studied (27%) had CPV titers below what is considered to be a safe titer for protection against CPV. And 25 of 117 dogs (21%) had titers below what is considered to be a protective titer for CDV.

As noted above, traditional methods to measure serum antibodies specific against CPV and CDV have included serum viral neutralization (SVN), hemagglutination inhibition (HI), enzyme-linked immunoassay (EIA or ELISA), immunofluorescence (IFA), and lateral flow immunoassay (LFIA), to name only a few. These assays are considered to be accurate, but are cumbersome, often require elaborate equipment, significant laboratory space, trained personnel, are expensive, and require venipuncture in order to collect the specimen to be run.

There are at least three tests presently marketed that are reported to work in clinic situations to check for CDV or CPV specific antibodies. However, these tests require serum, technical skill, time, and are expensive. One test by S. Y. Marulappa and S. Kapil (2008), reveals a method to test for CPV antigen in the feces as well as to measure CPV-specific antibodies. This test includes a cold glass slide with a circle to hold in the serum from the patient dilution of serum and raccoon parvovirus. This test is a modification of the hemagglutination inhibition (HI) test in which a toothpick is used to mix the reagents. Another test by the Synbiotics Corporation is called the “TiterCheck CDV/CPV” test and is advertised as a convenient in clinic qualitative assessment of antibody levels to canine distemper and canine parvovirus. This test is a microdot ELISA test that requires several steps and requires serum. It is a visual test and results are expected within 15-20 min.

A test disclosed by Waner (1996) is an ELISA developed to measure the serum decline in maternal antibodies which can interfere with vaccination by inactivating vaccine material before the puppy can interact with it.

A one-step chromatography assay test (LFIA) by Oh, et al. detects antibodies in the serum to CPV and has two lanes with which to compute the level of IgG antibody specific for CPV. This last test also requires serum to be drawn from a puppy, which can be traumatic for the owner, the puppy or adult dog, and can be frustrating for the clinician. This test gives two levels for serum IgG anti-CPV antibody.

By comparison, the subject test disclosed herein requires only saliva for the test and is more sensitive than that of Oh. et al. In addition, the subject test measures both IgA and IgG. The test measurement of IgA can show recent infection or vaccination with a modified live virus vaccine within 2-4 weeks of the exposure event and the test can simultaneously detect the levels of IgG in a more accurate fashion by having more detection lanes of bound anti-analyte antibodies.

It has been known for a long time that IgG has more avidity and affinity for an epitope than a competing IgA (Friedman, M, 1982). Therefore, the subject LFIA herein described contains an area located within the sample pad to bind to and remove from the flow the competing antibody from the reaction that the specific analyte is directed towards. If the analyte is specific IgA, then IgG is removed prior to contact with the analyte and ligand since IgG has a higher affinity and avidity for the competing epitope. Likewise, if the analyte is IgG, then the IgA is specifically removed prior to the IgG coming in contact with the ligand of the first reagent pad. IgA is present in much larger quantities than IgG and by mass action can compete for the ligand of the first reaction pad.

Again, current testing in laboratory situations to measure antibodies directed against CPV and CDV include serum virus neutralization (SVN), immunofluorescent antibody assay (IFA), enzyme-linked immunsorbent assay (ELISA), and hemagglutination inhibition (HI) and variations on these base tests. Laboratory testing while being the “gold standard” can be different depending upon the laboratory and method used. The testing in a central laboratory is also time consuming, expensive and labor and equipment intensive. Attempts have been made to make ELISA more adaptable to the clinic, but serum, expertise, time, and expense are still considerations. One LFIA test by Oh (2006), measures serum antibodies against CPV, but it is still invasive and does not measure antibodies to CDV.

Clearly, there is a need for an effective field testing kit for the screening of antibodies specific to CPV and CDV. A test that can be done while the patient waits, requires no more than a small volume of saliva, is inexpensive, and is repeatable is needed.

Concerning epidemiological vaccination studies, antibodies of the IgA, IgG, and IgM classes are found in saliva. The molecular weight of IgA is approximately 160 kDa and is transported into secretions and is found in saliva, tears, nasal secretions, tracheal secretions, urine, bile, and in blood serum. In saliva, IgA is preferentially transported through the epithelial lining by being connected to a secretory component (SC) which is about 50-90 kDa in size. Saliva with specific antibodies directed to various agents is well known in the human and veterinary literature. For example, IgA has been found to rise in the saliva after vaccination with a modified live virus vaccine. Also, IgA antibodies can act as effective virus-neutralizing agents (Tomasi, T. B, Ogra P. L. et al.). See Kerr, M. A., for a review of the structure and function of IgA.

Up to 80-90% of the IgA 1 subclass exists in monomeric form, while in external secretions up to 40% of the IgA belongs to the IgA 2 subclass and polymeric forms greatly predominate over monomers (Mestecky, J, et al., 1987). Additionally, in secretions such as saliva, IgA 2 subclass is in a dimeric form and is a polymeric secretory IgA that is bound with a J chain and also a heavily glycosylated protein call secretory component (SC). The SC molecule allows transcytosis of polymeric IgA and IgM to cross the epithelial barrier. SC is produced by epithelial cells lining the mucosal surface and is found in secretions complexed with IgA or IgM and also is a free glycoprotein. The purpose of SC is to increase resistance of the IgA molecule to proteolysis. The molecular weight of human SC can range somewhere between 50-90 kDa. Again, see Kerr, M. A. (1990) for a review of the IgA molecule and its role in the body.

Canine IgA is reviewed by Heddle & Rowley (1974) which revealed parotid gland salivary IgA values of 0.17-1.25 mg/ml and parotid gland salivary IgG concentrations of 0.005-0.05 mg/ml. Batt, et al. (1991) measured IgA in duodenal juice. German, et al. (1998) measured the production of both IgA and IgG by the method of ELISA, finding salivary IgA to be 682 EU/ml and salivary IgG to be 1.56 EU/ml. The values of Kikkawa (2003) showed that daily values are consistent, that the values rise during the morning until around noon time, and that stress reduces the amount of salivary IgA. For instance, Takahashi (2009), using an LFIA technique and comparing it to a commercial IgA test (Bethyl Labs, Tx), found that the minimum detection of IgA that could be measured at a 1:2500 dilution in PBS was 20 ELISA units ml⁻¹ (EUml⁻¹) and the maximum concentration was 140 EU ml⁻¹. ELISA values vary from laboratory to laboratory and are hard to standardize for these reasons.

Saliva makes an economical and noninvasive way to test for the presence of an analyte. Earlier work by Friedman (1982) showed that the response to mumps and mumps vaccine could be measured by following the salivary IgA response by radioimmunoassay (RIA). It was found that the IgA response could be detected as early as one day after the onset of illness, peaked at 1-2 weeks after exposure and persisted for 2-3 weeks after exposure. It was also found that it was important to use a specific binding agent for the IgA in order to avoid the over binding of IgG with its higher binding affinity and avidity.

Kinder (1992) using an ELISA method showed that both IgA and IgG responses to canine taeniasis could be measured in the saliva of dogs. Their work showed that in experimental infection salivary IgA rose with the magnitude of the egg counts while IgG was not raised. Saliva, then, becomes a useful method to assess the body response to infection. Sozemen, (Sozemen, et al., 1996) demonstrated that plasma cells secreting IgG, IgA, and IgM are located around salivary gland tissue, thus suggesting local production is also present.

A variety of methods have been disclosed to measure analytes in a body fluid such as saliva. Lateral flow immunoassays have become very numerous in the last several years. These tests provide quick and fairly accurate results on a wide range of diagnostic problems and at an affordable price. Many of these tests have become patented or are in the open literature. References to liposomes as the reporter have been disclosed (4636479). A method of measuring salivary secretory IgA in dogs has been published showing the feasibility LFIA in this setting (Takahashi, A.). In this method salivary IgA was estimated to be between 20 and 140 EU/ml based upon an ELISA standard. Eisinger (4943522) showed that the kinetics of the lateral flow technique were improved with the non-bibulous material Porex, which allowed all molecules to travel at the same rate. Pawlak, et al. (WO 92/12428) improves upon the work of Eisinger by making a bibulous support into a nonbibulous lateral flow strip by blocking it. This work by Pawlak is hereby incorporated by reference. The principle advantage is a test that is much faster than using bibulous support. Such blocking agents include bovine serum albumin (BSA), methylated-BSA, succinylated-BSA, whole serum from horse, fetal calf serum, or other blood products or non-fat dried milk. Non-fat dried milk has long been used in the ELISA process to block excess binding sites. Certain detergents or long-chain alcohols such as PVA can also be used (Bruning, 1999).

### BRIEF SUMMARY OF THE INVENTION

An object of the present invention is to present an apparatus having one or more lateral flow immunoassay test channels in a test cassette for the detection of one or more desired analytes. In particular, the subject system detects antibodies IgA and/or IgG found in canine saliva to canine parvovirus (CPV) and/or canine distemper virus (CDV) by means of selectively identifying desired species via chromatographic techniques after their migration down a membrane support within the cassette. The analytes (IgA and/or IgG) migrate through the membrane support and are detected by a concentrated presence of bound gold nanoparticles at designated sites on the test cassette.

Another object of the present invention is to provide a test strip with a zone where a detectable tracer, in this zone, is also mobilizable, with a first immobilized binding partner for an analyte and a binding affinity for the detectable tracer and an area wherein a second immobilized binding partner has an affinity for the first tracer. This embodied method allows the binding of biotin with avidin which is separate from and just downstream and a second conjugation pad made of an adsorbent material of fiberglass or other like fiber in which the adsorbed biotin is linked to gold nanoparticles (gnp residue) or the equivalent. Further, the reservoir is in a separate area from the inlet/port and is made of a sorbent material such as fiberglass or the equivalent. The reservoir acts to receive, to mix, and to redirect the liquid with the conjugates along a flow path to, in, and along a material which is non-permeable to the capture zones and to the control capture area. An absorbent pad in contact with the non-permeable strip then wicks excess fluid away from the reaction sites. A set of colored lines are then visually read and interpreted as to the status of the sample.

Yet another object of the present invention is to furnish a detection system and method of use in which antibodies of the IgA and IgG classes when present in saliva from a dog vaccinated against canine parvovirus bind to canine parvovirus. As part of the formulation, a subject test kit provides gold nanoparticles labeled species. Antibodies made in another species and directed against the Fc portion of each class of antibody can bind the Fc portion of the IgA or IgG molecules. When the Fc portion of the molecule is bound, the complex of either IgA or IgG complexed to the canine parvovirus label is bound. The gold nanoparticles concentrate at the site rendering a red line for detection.

Disclosed is a test apparatus, test system, and method of use employing lateral flow immunoassay (IFA) procedures for the detection of a selected ligand in a liquid sample from a body fluid of dog saliva (the canine example is utilized as an exemplary species for the subject application). The subject invention test system includes a port for a sample to enter onto a sample pad which filters out debris and has, usually, bound antibodies for removing any interfering glycoproteins and interfering antibodies in the sample. The possible contaminating antibody problem is resolved in the subject invention by the removal of the competing antibody prior to the first conjugation step in the subject testing system. The sample pad is in contact with a permeable strip of nitrocellulose or similar/equivalent material. Next to the sample pad and attached to a blocked permeable membrane is a first conjugation pad made of fiberglass or similar/equivalent material. Adsorbed in the first conjugation pad, but not fixed, is the first conjugation reagent that is bound to biotin. A method is related for providing a test strip with a zone where a detectable tracer, in this zone, is also mobilizable, with a first immobilized binding partner for a desired analyte and a binding affinity for the detectable tracer and an area wherein a second immobilized binding partner has an affinity for the first tracer. This subject method allows the binding of biotin with avidin which is separate from and just downstream and a second conjugation pad made of an adsorbent material of fiberglass, or other equivalent material, in which the adsorbed biotin is linked to gold nanoparticles (gnp residue). Further, the reservoir is in a separate area from the inlet and is made of a sorbent material such as fiberglass or the equivalent. The reservoir acts to receive, to mix, and to redirect the liquid with the conjugates along a flow path to, in, and along a material which is non-permeable to the capture zones and to the control capture area. An absorbent pad in contact with the non-permeable strip then wicks excess fluid away from the reaction sites. A set of colored lines are then visually read and interpreted as to the status of the sample.

Additionally, antibodies of the IgA and IgG classes when present in saliva from a dog vaccinated against canine parvovirus bind to the canine parvovirus. As part of the subject formulation, a subject test kit provides gold nanoparticles labeled species. Antibodies made in another species and directed against the Fc portion of each class of antibody can bind the Fc portion of the IgA or IgG molecules. When the Fc portion of the molecule is bound, the complex of either IgA or IgG complexed to the canine parvovirus label is bound. The gold nanoparticles concentrate at the site rendering a red line for detection. The same reaction and end point are also detected when CDV antigen is substituted for CPV.

Further, the subject invention is an apparatus, system, and process to assay for an analyte or analytes. The subject process comprises one or more analytes in a sample contacting a sample port (single test-lane embodiment) or ports (a plurality of test-lanes embodiment) in a test cassette, wherein the sample port or ports are connected to a sample pad or pads of absorbent material which optionally filters the applied sample(s) in which the analyte or analytes reside(s) and may optionally contain binders to remove cross reacting contaminants in the sample solution(s) such as non-analyte proteins like cross-reacting antibodies in the saliva. The sample pad is in contact with a strip of material that allows lateral flow. The open areas of the strip, that is, the areas wherein there are no fixed binding ligands (“open”) or other binder(s) thereto associated, are blocked with appropriate blocking agents to prevent binding. The first reagent pad is in contact with the strip. This first reagent pad contains unfixed ligand to which the analyte will bind or proteins such as monoclonal antibodies (Mabs) or adsorbed polyclonal antibodies (Pabs) which will compete with the analyte for a ligand depending upon the presentation. The ligand or competing analyte of conjugate pad 1 (1^{st} tracer) is coupled with biotin (biotinylated). The strip is made of various materials including permeable materials, non-permeable materials, and equivalent materials. The permeable membrane can be blocked to make it non-permeable and to improve flow characteristics. Downstream, and in contact with the lateral flow strip, is a second permeable pad, conjugate pad 2, made of a material such as fiberglass or synthetic fiber and contains an unfixed ligand or ligands (2^{nd} tracer or tracers). The second conjugate pad (pad 2) is composed of a ligand or ligands not binding to the analyte. The second conjugate pad is composed of unbound avidin bound to, and labeled with, a visible or measurable particulate label or labels that bind(s) to the biotin of the 2^{nd} conjugate pad. The label (“label”) being composed of a number of visible agents such as from a group which may include, but not limited to, nanoparticles of metals such as gold or silver, carbon nanoparticles, liposomes, colored beads such as plastics like polystyrene, or dyes or cells which are themselves labeled. Analyte and first ligand from the first conjugate pad combine and then come into contact with the conjugate from the second pad and form a further conjugate. As the fluid/solvent front moves downstream on the strip the analyte-conjugate 1-conjugate 2 comes into contact with bound tracer or tracers directed against analyte fixed into the non-permeable strip. These bound tracers then form a further complex, concentrate the visible reader from conjugate pad 2, and form a visible mark such as a detectable line. The conjugate then may continue downstream and come in contact with a second, or third, or fourth, or fifth or more binder which is fixed to the permeable strip in appropriate contact regions. A complex at each contact region forms between first tracer, analyte, and second tracer to form a visible or measurable complex. Lines or marks of bound tracers to which the conjugate may bind may include any agent that will bind to the analyte including, but not limited to, antibodies, parts of antibodies, protein A, protein G, jacalin, or any other binder of the analyte and may be made of a monoclonal antibody (Mab) or a polyclonal antibody (Pab) made in a mammal such as a goat, fowl, or equivalent animal or in vitro environment. Just distal or downstream from the last conjugate binding site on the lateral flow strip is a control line of bound tracer directed toward the second conjugate such that when binding takes place forms a visible complex. The remainder of the unbound fluid then flows into a bibulous absorbent pad that is in contact with the distal end of the lateral flow strip. The entire strip or strips is/are housed in a plastic test cassette within, usually, a protective sealed bag with a desiccant.

Further objects and aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a cross-sectional side view of a single test-lane embodiment of the subject invention.

FIG. 2 is top view of a four test-lane embodiment of the subject invention.

### DETAILED DESCRIPTION OF THE INVENTION

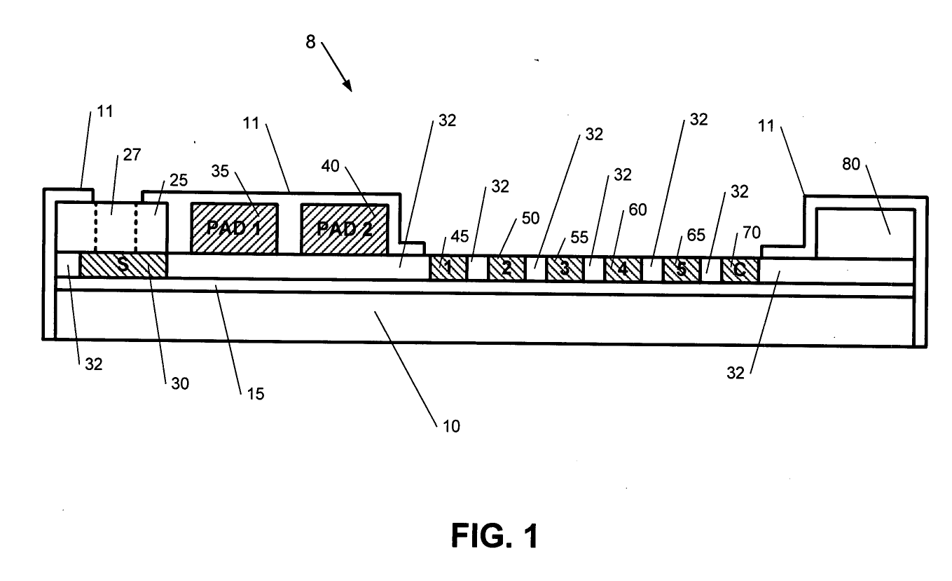

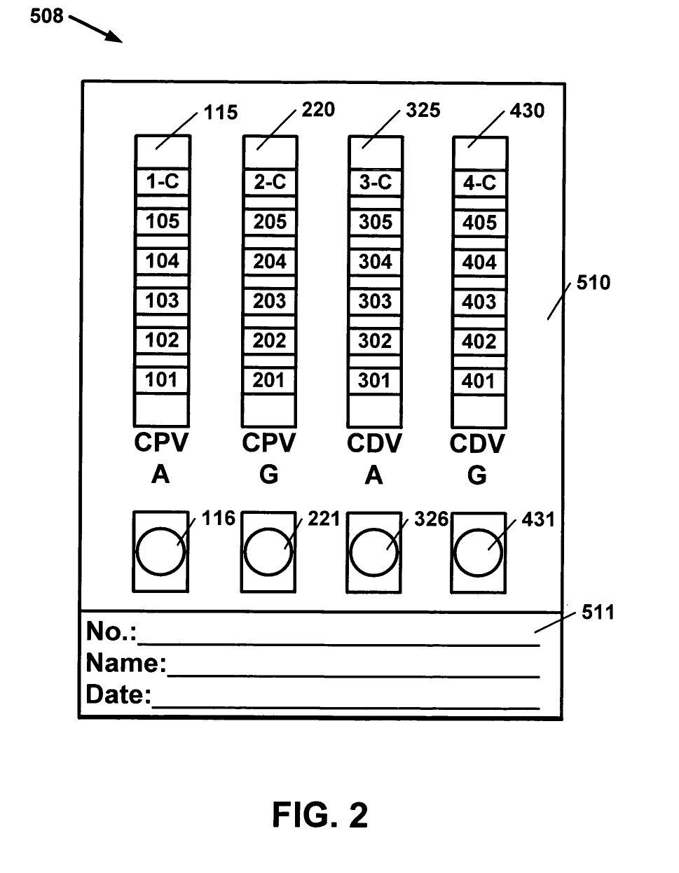

Referring more specifically to the drawings, for illustrative purposes the present invention is embodied in the apparatus generally shown in FIGS. 1 and 2. It will be appreciated that the apparatus may vary as to configuration and as to details of the parts, and that the method may vary as to the specific steps and sequence, without departing from the basic concepts as disclosed herein.

Generally, the subject invention utilizes one or more lateral flow immunoassay test channels in a test cassette for the detection of one or more desired analytes. In particular, the subject system detects antibodies IgA and/or IgG found in canine saliva to canine parvovirus (CPV) and/or canine distemper virus (CDV) by means of selectively identifying desired species via chromatographic techniques after their migration down a membrane support within the cassette. The analytes (IgA and/or IgG) migrate through the membrane support and are detected by a concentrated presence of bound gold nanoparticles at designated sites on the test cassette.

The subject invention, including the subject apparatus, system, and process, concerns assaying for an analyte or analytes in canine saliva. The subject test apparatus may be fabricated with a single test channel 8 (see FIG. 1) or with multiple test channels 508 (see FIG. 2). The subject process comprises one or more analytes in a sample S contacting a sample port 27 (in the single channel embodiment in FIG. 1) or ports 116, 221, 326, and 432 (in the multiple channel embodiment in FIG. 2) in a test cassette, wherein the sample port 27 or ports 116, 221, 326 and 431 are connected to a sample pad 30 or pads (not shown in the multiple channel embodiment) of absorbent material which optionally filters the applied sample(s) in which the analyte or analytes reside(s) and may optionally contain binders to remove cross reacting contaminants in the sample solution(s) such as non-analyte protein such as cross-reacting antibodies in the saliva. A suitable solvent/solution is added to the sample pad 30 and utilized to wash the sample S downstream.

As seen in FIG. 1, the subject single channel cassette is comprised of a supporting base member 10 and a surrounding upper body shell 11 with appropriate apertures for sample application 27 and analyte detection presentation regions 45, 50, 55, 60, 65, and 70. As seen in FIG. 2, the subject multiple channel embodiment is comprised of a supporting base member (not directly seen) and a surrounding upper body shell 510 with appropriate analyte detection presentation regions comprising: a first channel 115 with regions 101, 102, 103, 104, and 105 (or fewer or greater, if desired) and control aperture 1-C; a second channel 220 with regions 201, 202, 203, 204, and 205 (or fewer or greater, if desired) and control aperture 2-C; a third channel 325 with regions 301, 302, 303, 304, and 305 (or fewer or greater, if desired) and control aperture 3-C; and a forth channel 430 with regions 401, 402, 403, 404, and 405 (or fewer or greater, if desired) and control aperture 4-C. It is stressed that the multiple channel embodiment may have two or more channels and the four channel embodiment seen in FIG. 2 is only by way of example and not by way of limitation.

As seen clearly in FIG. 1 (with the equivalent structures also present in the multiple channel embodiment, but not directly seen in the top view seen in FIG. 2), the sample pad(s) 30 is in contact with a strip of material 32 that allows lateral flow of liquid/solvent. A support layer 15 is frequently below the strip 32 and above the support base 10. The open areas of the strip 32 and between the detection display apertures, that is, the areas wherein there are no fixed binding ligands (“open”) or other binder(s) thereto associated, are blocked with appropriate blocking agents to prevent binding. The first reagent pad 35 (Pad-1) is in contact with the strip 32. This first reagent pad 35 contains unfixed ligand to which the analyte will bind or proteins such as monoclonal antibodies (Mabs) or adsorbed polyclonal antibodies (Pabs) which will compete with the analyte for a ligand depending upon the presentation or assay configuration. The ligand or competing analyte of conjugate Pad-1 35 (1^{st} tracer) is coupled with biotin (biotinylated). The strip 35 is fabricated from various materials including nitrocellulose (permeable), Porex (non-permeable) (Porex Technologies, Fairburn, Ga., USA), and equivalent materials. The permeable membrane strip 32 can be blocked in selected regions to make it non-permeable and to improve flow characteristics. Downstream, and in contact with the lateral flow strip 32, is a second permeable pad 40, conjugate Pad-2, made of a material such as fiberglass or synthetic fiber and contains an unfixed ligand or ligands (2^{nd} tracer or tracers). Conjugate Pad-2 is composed of a ligand or ligands not binding to the analyte. Further, conjugate Pad-2 is composed of unbound avidin bound to, and labeled with, a visible or measurable particulate label or labels that bind(s) to the biotin of conjugate Pad-1. The label (“label”) being composed of a number of visible agents such as from a group which may be included, but not limited to, nanoparticles of metals such as gold or silver, carbon nanoparticles, liposomes, natural or synthetic polymers such as colored beads such as plastics like polystyrene, or dyes or cells which are themselves labeled. Analyte and first ligand from conjugate Pad-1 35 combine and then come into contact with the conjugate from Pad-2 40 and form a further conjugate (analyte-conjugate-1-conjugate-2).

As the fluid/solvent wave moves downstream on the strip 32 the analyte-conjugate-1-conjugate-2 comes into contact with bound tracer or tracers fixed to the strip or immediately available to the strip 32. These bound tracers then form a further complex, concentrate the visible reader from conjugate pad 2, and form a visible mark such as a detectable line that the user is able to see. After the first bound tracer is encountered, the conjugate excess then may continue downstream and come in contact with a second, or third, or fourth, or fifth or more binder which is fixed to the permeable strip in appropriate contact regions (1, 2, 3, 4, and 5 for the single lane embodiment as seen in FIGS. 1 and 101, 201, 301, 401, 102, 202, 203, 204, 205, 103, 203, 303, 403, 405, 104, 204, 304, 404, and 105, 205, 305, 405 for the a multiple lane embodiment as seen in FIG. 2). A complex at each contact region forms between first tracer, analyte, and second tracer to form a visible or measurable complex. Lines or marks of bound tracers to which the conjugate may bind may include any agent that will bind to the analyte including, but not limited to, antibodies, parts of antibodies, protein A, protein G, jacalin, or any other binder of the analyte and may be made of a monoclonal antibody (Mab) or a polyclonal antibody (Pab) made in a mammal such as a goat, fowl, or equivalent animal or in vitro environment. Just distal or downstream from the last conjugate binding site on the lateral flow strip is a control line (C in FIG. 1 for the single lane embodiment and 1-C, 2-C, 3-C, and 4-C for the multiple lane embodiment seen in FIG. 2) of bound tracer directed toward the second conjugate such that when binding takes place forms a visible complex. The remainder of the unbound fluid then flows into a bibulous absorbent pad 80 that is in contact with the distal end of the lateral flow strip 32.

Additionally, the subject invention comprises an analyte detection system/apparatus and method of use in which selected saliva is tested for the presence of antibodies directed against infectious species. The test apparatus is comprised of a cassette which has either a single test-lane (FIG. 1) or a plurality of test-lanes (FIG. 2). Specifically, FIG. 1 depicts a single test-lane embodiment and FIG. 2 shows a four test-lane embodiment. Each test apparatus (single-lane 8 or multi-lane embodiments 508) is housed in a surrounding plastic (or equivalent) test cassette/enclosure. Specifically, for the single test-lane embodiment 8 seen in FIG. 1, the device base 10, usually fabricated from a plastic material or the equivalent, extends into a surrounding case 11 that is, also, normally plastic or the equivalent. When sold or distributed, the device is normally packaged within a protective sealed bag with a desiccant and/or preservative material.

For a sample S of saliva, the saliva is applied to the device via an aperture/port 27 in the single lane embodiment cassette and loaded onto a filtering composite membrane 30 where contaminating particulate matter and test-interfering antibodies are removed or trapped. Thus, the subject invention test system includes a port 27 for a sample S to enter onto a sample pad 30 which filters out debris and has suitable bound antibodies for removing any interfering glycoproteins and interfering antibodies in the sample. Friedman, M. (1982) showed that the presence of IgG in samples can interfere with the binding of IgA, because of its greater avidity of binding. Similarly, in saliva, the large amount of IgA relative to IgG might block the binding sites of the ligand preventing the binding of IgG. This problem is resolved in the subject invention by the removal of the competing antibody prior to the first conjugation step in the subject testing system.

The solution being tested S then travels along a non-permeable 32 membrane where it comes in contact with Pad-1 35 where there is provided an agglutination ligand capable of agglutinating with the analyte. In the subject method the analyte being measured comes in contact with a suspension of agglutinatable particles, ligand, having a surface bound biotin molecule. The conjugate therein formed next is allowed to react with a Pad-2 40 containing a second agglutinatable solution that contains the reporter group avidin attached to gold nanoparticles. At this point, a further agglutination then takes place.

Further, in another embodiment of the subject invention, a competitive assay is performed. Saliva is placed on the first filtering pad S, as above, and any particulate material and interfering antibodies are removed. The analyte then moves down the membrane 32 until it mixes, by wetting, with an analyte analogue in Pad-1 35. The analyte mixes with the analogue which agglutinates with the bound tracer. The analogue is biotinylated. The two, analyte and analogue bound to biotin then flow to Pad-2 where avidin-labeled gold nanoparticles are located. The analogue-biotin combines with the avidin-gold nanoparticles to form a large conjugate relative to the analyte. The analyte then flows through fixed lines or lanes of bound ligand of different concentrations (1, 2, 3, 4, and 5 for the single lane embodiment as seen in FIGS. 1 and 101, 201, 301, 401, 102, 202, 203, 204, 205, 103, 203, 303, 403, 405, 104, 204, 304, 404, and 105, 205, 305, 405 for the a multiple lane embodiment as seen in FIG. 2). The analyte binds to or competes with the analogue for open surfaces of the bound ligand preventing colored complex of the analogue from binding. The different concentrations of ligands represent a semiquantitative measure of analyte present based upon the lack of color from the analogue conjugate from binding to the early lanes of bound ligand. A control lane(s) (C in FIG. 1 for the single lane embodiment and 1-C, 2-C, 3-C, and 4-C for the multiple lane embodiment seen in FIG. 2) composed of biotin bound to the membrane 32 serves as a visual marker that the fluid has moved past the bound ligand. Again, the bibulous pad 80 in contact with the strip 32 wicks excess fluid/solvent away.

### EXAMPLES

CPV virus ligand is grown after the method of Oh (2006). CRFK cells (CCL-94; ATCC) are grown in Dulbecco modified Eagle medium (catalog no. 12100-046; Gibco) with 10% fetal calf serum (FCS) and gentamycin antibiotics until reaching a monolayer stage. C-780916 strain (VR-953: ATCC) are inoculated and grown in the CRFK cells with 2% FCS. The cells are grown until cytopathic effect (CPE) is noted, approximately 3-4 days. The cells are frozen and thawed 3 times. The harvested fluid is then centrifuged at 500×g in the cold for 15 minutes to remove large cellular debris. Formaldehyde was used by Oh, however, beta-propiolactone (BPL) has been shown to degrade the HA antigens less (Pollock, R V and Carmichael, L. E, 1982). BPL is used here for the reason just cited. The CPV, which is now inactivated is treated with polyethylene glycol 6000 (Sigma, St. Louis, Mo.) as described after Adams, 1973. The solution is allowed to stand in 0.4-0.6 M sodium chloride. The resulting solution is then centrifuged and the pellet resuspended in Tris-chloride buffer with sodium azide until further use.

An alternative method to grow CPV is disclosed by Hummel, K. (1992), Saliki, et al, (1992), and Ismail, et al, (1994), in which the CPV structural protein VP-2 gene sequences are fused to baculovirus polyhedron promoter. The baculovirus is then used to infect insect cell lines, Sf9, and the larvae (Spodoptera fruqiperda). Crude extracts provide VP-2 protein for binding to biotin.

The method of preparation of CPV viral particles enriched for VP-2 protein by biotinylation is after the method of Anderson (1986) and herein referenced (all noted references are herein incorporated by reference). The protein is dialyzed against 0.1M NaHCO₃ (pH8.4). After dialysis adjust the protein concentration to 1 mg/ml. One ml of 1 mg/ml protein solution is then reacted with 0.2 ml of DMSO and 1.4 mg of N-biotinylated-w-aminocaproic acid-N-hydroxysuccinimide ester for 4 hours. The resulting solution is dialyzed against 0.01M phosphate buffered saline (PBS, pH7.2) overnight at 4° C. The solution is stored at −20° C.

Gold nanoparticles (gnp) conjugated to avidin are provided by various suppliers (Cytodiagnostics, Pierce). Gnps are conjugated to avidin after the method of Cytodiagnostics, Bulletin #102. Briefly, 250 μl of gnp are transferred from stock and place in Eppendorf tubes. The pH of the gold colloid is adjusted to match the pl of the protein to be conjugated. An amount of up to 1 mg of protein in 25 μl is added to the gnp while mixing to titrate the amount needed to saturate the gold surface. The mixture is incubated for 2-3 minutes at room temperature. Then, 250 μl of 10% NaCl solution is added. A color change is observed and a determination is made as to which protein concentration the gold nanoparticles surface is saturated and no aggregation occurs upon addition of 10% NaCl. This can be observed by an increase in absorbance at 580 nm compared to a control.

The labeled avidin is supplied by Cytodiagnostics, Burlington, Ontario, Canada. Monoclonal antibodies (Mabs) and polyclonal antibodies (Pabs) against IgA Fc are purchased (Bethyl Labs. TX, USA). Polyclonal antibodies (Bethyl Labs, Tx) are affinity purified to be canine IgG or IgA Fc specific. Five lanes of anti-canine IgA or IgG Fc specific antibodies are placed with approximately a width of 0.7-1.0 mm transverse to the long axis of the lateral flow membrane.

The construction of the strip is as follows and is hereby referenced from Takahashi (2009). Pab-canine IgA is made to a concentration of 1 mg/ml in PBS, pH 7.2. The base solution for binding to the nitrocellulose strip as follows. The Pab, 650 μl was mixed with 50 μl 20% sucrose diluted in 50 mM KH₂PO₄ (pH7.5) and 50 μl of 2-propanol. Serial dilutions by 2 were prepared in a checkerboard fashion to match paired serum samples to match serum samples. A typical lane represents a 1:80 HI dilution for CPV and a 1:20 for CDV. The control line is prepared by mixing BSA-avidin at a concentration of 1 mg/ml 40 μl with 60 μl of 2-propanol and 1100 μl of 50 mM KH₂PO₄ buffer (pH 7.5).

The above solutions are applied to the nitrocellulose strip with a dispensing system (Biojet Quanti 300, BioDot, Inc. CA, USA). After dispensing and drying and fixing of the lanes, the open sites are blocked after the method of Pawlak, WO 92/12428 and Pronvost, 6656744, and hereby referenced in their entirety and by Bruning (1999).

Blocking of the nitrocellulose open sites is accomplished by the method of Bruning (1999). In short, the nitrocellulose sheet is air-dried for 10 minutes or until dry. Blocking is then accomplished by soaking in 1% polyvinyl alcohol (PVA) solution for 30 minutes. The paper is then washed twice for 15 minutes with distilled water, allowed to air dry, lyophilized, and then cut into strips of 5×60 mm.

Square fiberglass filter sheets (Whatman) are soaked in one of the following solutions, processed, and handled in the following manner. Squares, as outlined, are treated with a solution of 100 μl per 30 cm of fiberglass of conjugates 1 or 2 dissolved within 0.1M Tris-sucrose buffer, pH 8.0. The sheets are then dried at room temperature for 45 minutes or until dry.

Each sheet is treated as above with a solution of biotinylated CPV or CDV at various concentrations in a checkerboard fashion. These squares are designated as conjugate Pad-1 and are then cut into 5×5 mm squares, treated with adhesive and then placed as shown in the figures.

Similarly, squares, as outlined above, are soaked in avidin-gnp solution. These squares are designated conjugate Pad-2. A checkerboard arrangement is set up in order to assess the best range of final concentrations. Porex fiber sample pads are made by soaking and binding Pab anti-canine IgG into the pad sheets. The Pab anti-canine IgA or IgG Fc specific antibodies are dried, lyophilized, and stored until use. These pads are then cut into 5×5 mm squares, dried, and adhered to the front of the blocked nitrocellulose sheet with adhesive. Bibulous paper (Whatman) is cut into 5×15 mm rectangles and place onto the downstream end of the nitrocellulose strip at the end of the strip.

In another embodiment, a competitive method is set up in which a Mab directed against CPV or CDV is generated. The method of Adler and Faine (1983), Farrelly (1987) and Riddell et al (2000) is hereby referenced for the production of Mabs. The use of Mabs is also referenced in use for LFIA's, the patents of Valkirs (4632901). Specific Mabs are purchased from various vendors such as Bethyl Labs (TX, USA). Mabs are produced in the following manner: eight-week-old BALB/c mice are immunized at 0 and 4 weeks by intraperitoneal inoculation with equal volumes of purified CVP VP2 antigen or with CDV N antigen at 100 μg in 100 μl and Hunter's Titermax (Sigma Chemicals, St. Louis, Mo., USA). After 4 weeks, a final injection of equal volumes of antigen and 0.85% saline are injected in the tail vein. Three days later 10-8^{th} mouse spleen cells are fused with 10-9^{th} mouse myeloma cells sp2/0-Ag 14 or NS-1 using polyethylene glycol 1500 (50%, wt/vol), Sigma (St. Louis, Mo., USA) in Dulbecco Modified Eagle's Medium (DME, Microbiological Assoc, Bethesda, Md., USA) after the method of Kolher and Milstein (1975). Limiting dilution of cells into culture is made. Resultant hybridomas are grown and screened by MAT or by EIA and cloned by limiting dilution. All hybridomas are cloned at least twice and then grown to a volume of approximately 1000 ml in DME. Supernatant is harvested, centrifuged, and concentrated. The final antibody concentration is maintained at 0.5-1.0 mg/ml. The portion used in competitive assay is used biotinylated as previously described.

Specific Pab anti-canine CPV or CDV (Bethyl Labs, TX) is biotinylated as above. Biotinylation takes place as discussed above. This biotinylated solution is then place into fiberglass sheets and processed as described above. These sheets are designated as conjugate Pad-1, are then cut into squares as above, and placed upon the lateral flow strips.

Avidin-gnp squares are treated as above and are designated conjugate Pad-2. These pads are cut into 5×5 mm squares and are placed in position distal to conjugate Pad-1.

For example, but not by way of limitation, usually, five lines of bound ligand are placed upon each nitrocellulose sheet. The lines are approximately 0.7-1.0 mm in width and are placed equidistant from each other and between the conjugate pad 2 and distal absorbent pad locations. The sheets are then fixed, dried, blocked and then lyophilized. The sheets are then cut into 5×60 mm strips. A sample port is adhered to the upstream end, conjugate Pad-1 is then adhered on followed by the conjugate Pad-2 and then by the distal absorbent pad. A waterproof sheet is then placed under the strip and a cardboard or plastic backer is placed under the waterproof sheet (support layer 15 in FIG. 1). A cover with hole for sample placement and a viewing box is place over the top of the strips and is glued to the bottom support or snaps into place if both are of formed plastic. The final assembly is completed as in seen in FIG. 1 for the equivalent single channel embodiment and in FIG. 2 for the equivalent multiple channel embodiment (see further above for exact numbered components).

### Comparison of Disclosed Method to Standardized Tests

Paired samples of serum are collected with each sample of saliva. The centerline for each IgG of each set of 5 bound ligands is diluted to represent the accepted standard for the minimum of protection for that particular virus as measured in serum. For example, the center line for CPV for the IgG will be set as if the serum is HI of >1:80 reciprocal dilution, (McCaw, 1998). The reference here to HI titre is because this test is one of the most accepted laboratory tests regarding CPV. Saliva titrations will be set for the centerline and will be prediluted accordingly by a checkerboard titration.

CDV titres for IgG are compared to SVN titres similarly because this is a recognized and accepted test for CDV. Titres of >1:96 are considered indicative of protective titres against CDV (McCaw, 1998). Saliva IgA is evaluated compared to a range of values from dogs naive to vaccination and results serially taken after vaccination with a modified live virus vaccine. Similarly, saliva titrations will be set for centerline and will be prediluted accordingly by a checkerboard titration.

In one embodiment, a cassette to measure antibodies to CPV will consist of two parallel strips, one for specific IgA and one for specific IgG. Puppies with vomiting and diarrhea and an unknown vaccination history can now be checked with saliva. A low level of specific IgA and a high level of specific IgG suggests that CPV is not the problem while a high specific IgA anti-CPV and a low specific IgG anti-CPV would suggest recent viral exposure. Appropriate decisions and therapy could then be taken as needed.

In another embodiment, a cassette to measure CDV will consist of two parallel strips, one for specific IgA anti-CDV and one for specific IgG anti-CDV. A low level of specific IgA and a high level of specific IgG suggests that CDV is not the problem while a high specific IgA anti-CDV and a low specific IgG anti-CDV would suggest recent viral exposure, Appropriate decisions and therapy could then be taken as needed.

In still another embodiment, four parallel strips will measure canine antibodies to both CDV and CPV. Such a strip can be used where dogs are brought together such as humane facilities. In this context, a high saliva titer may represent recent viral exposure or a high IgG titer may represent adequate protection. Such dogs could then be further segregated, observed, and/or vaccinated as needed. Dogs with high titres of IgG would not require vaccination, thus, saving time, effort, money, and potential for over stimulation of the body's immune system.

Table 1 (immediately below) shows three sets of conditions with each set representing a specific testing goal: Goal of Set #1 is to measure saliva antibodies against CVP and CDV via the direct method (all Pab and Mab are Fc specific); Goal of Set #2 is to measure saliva antibodies against CVP and CDV via the competitive method (all Pab and Mab are Fc specific); and Goal of Set #3 is to measure saliva antibodies against CVP and CDV via a double sandwich assay.

TABLE 1
CANINE SALIVARY IgA/IgG ANTIBODIES AGAINST CANINE
PARVOVIRUS (CPV AND CANINE DISTEMPER VIRUS (CDV)

| | | SAMPLE | | | BOUND | |
|---|---|---|---|---|---|---|
| | ANALYTE | PAD | CONJUGATE 1 | CONJUGATE 2 | LIGAND | CONTROL |
| | GOAL: MEASURE SALIVA ANTIBODIES AGAINST CVP AND CDV VIA THE | | | | | |
| | DIRECT METHOD (all Pab and Mab are Fc specific) | | | | | |
| SET #1 | IgA | Pab anti- | CPV/VP2- | avidin-gnp | 1. Pab anti- | biotin |
| | | IgG | biotin | | IgA | |
| | | | | | 2. Mab anti- | |
| | | | | | IgA | |
| | | | | | 3. Protein A | |
| | | | | | 4. anti-SC | |
| | IgG | Pab anti- | CPV/VP2- | avidin-gnp | 1. Pab anti- | biotin |
| | | IgA | biotin | | IgG | |
| | | | | | 2. Mab anti- | |
| | | | | | IgG | |
| | | | | | 3. Protein A | |
| | IgA | Pab anti- | CDV N- | avidin-gnp | 1. Pab anti- | biotin |
| | | IgG | protein-biotin | | IgA | |
| | | | | | 2. Mab anti- | |
| | | | | | IgA | |
| | | | | | 3. Protein A | |
| | | | | | 4. anti SC | |
| | IgG | Pab anti- | CDV N- | avidin-gnp | 1. Pab anti- | biotin |
| | | IgA | protein-biotin | | IgG | |
| | | | | | 2. Mab anti- | |
| | | | | | IgG | |
| | | | | | 3. Protein A | |
| | GOAL: MEASURE SALIVA ANTIBODIES AGAINST CVP AND CDV VIA THE | | | | | |
| | COMPETITIVE METHOD (all Pab and Mab are Fc specific) | | | | | |
| SET #2 | IgA | Pab anti- | IgA anti-CVP- | avidin-gnp | CVP/VP2 | biotin |
| | | IgG | biotin | | prot | |
| | IgG | Pab anti- | IgG anti- | avidin-gnp | CVP/VP2 | biotin |
| | | IgA | CVP-biotin | | prot | |
| | IgA | Pab anti- | IgA anti-CDV- | avidin-gnp | CDV N-prot | biotin |
| | | IgG | biotin | | | |
| | IgG | Pab anti- | IgG anti- | avidin-gnp | CDV N-prot | biotin |
| | | IgA | CDV-biotin | | | |
| | GOAL: MEASURE SALIVA ANTIBODIES AGAINST CVP AND CDV VIA A | | | | | |
| | DOUBLE SANDWICH ASSAY | | | | | |
| SET #3 | IgA | Pab anti- | Mab anti-IgA | avidin-gnp | CPV/VP2 | biotin |
| | | IgG | Fc-biotin | | prot | |
| | IgG | Pab anti- | Mab anti-IgG | avidin-gnp | CPV/VP2 | biotin |
| | | IgA | Fc-biotin | | prot | |
| | IgA | Pab anti- | Mab anti-IgA | avidin-gnp | CDV N-prot | biotin |
| | | IgG | Fc-biotin | | | |
| | IgG | Pab anti- | Mab anti-IgG | avidin-gnp | CDV N-prot | biotin |
| | | IgA | Fc-biotin | | | |

Various claimed embodiments of the subject invention are herein related immediately below:

A lateral flow immunoassay (LIFA) apparatus for detection of an analyte in a body fluid sample from an animal pet, utilizing a test cassette, comprised of: a) a test cassette housing; b) a sample port formed in said test housing that accesses a sample pad for receiving the body fluid sample c) a strip of material that allows lateral flow of liquid and is fitted within said cassette housing, wherein said strip of material is in contact with said sample pad; d) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate; e) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate; f) a first tracer affixed to said strip in an analyte detection zone that is downstream from said second permeable conjugate pad, wherein said tracer forms a detectable complex producing a visible mark upon encountering said third conjugate in said analyte detection zone; g) a control zone downstream of said analyte detection zone, wherein said control zone comprises bound a second tracer directed toward said second permeable conjugate pad unfixed ligand and forms a visible complex upon binding; and h) an absorbent pad that is in contact with the distal end of said strip for absorbing liquid from said strip.

A lateral flow immunoassay (LIFA) apparatus for detection of an analyte in a sample of saliva from a dog, wherein exposure to a virus selected from a group consisting of canine parvovirus (CPV) and canine distemper virus (CDV) is detected, utilizing a test cassette, comprised of: a) a test cassette housing; b) a sample port formed in said test housing that accesses a sample pad for receiving the saliva sample; c) a strip of material that allows lateral flow of liquid and is fitted within said cassette housing, wherein said strip of material is in contact with said sample pad; d) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate; e) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate; f) a first tracer affixed to said strip in an analyte detection zone that is downstream from said second permeable conjugate pad, wherein said tracer forms a detectable complex producing a visible mark upon encountering said third conjugate in said analyte detection zone; g) a control zone downstream of said analyte detection zone, wherein said control zone comprises bound a second tracer directed toward said second permeable conjugate pad unfixed ligand and forms a visible complex upon binding; and h) an absorbent pad that is in contact with the distal end of said strip for absorbing liquid from said strip.

A method utilizing lateral flow immunoassay (LIFA) for detecting an analyte in a body fluid sample from an animal pet, comprising the steps of: a) providing a test cassette, wherein said test cassette comprises: i) a test cassette housing; ii) a sample port formed in said test housing that accesses a sample pad for receiving the body fluid sample; iii) a strip of material that allows lateral flow of liquid and is fitted within said cassette housing, wherein said strip of material is in contact with said sample pad; iv) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate; v) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate; vi) a first tracer affixed to said strip in an analyte detection zone that is downstream from said second permeable conjugate pad, wherein said tracer forms a detectable complex producing a visible mark upon encountering said third conjugate in said analyte detection zone; vii) a control zone downstream of said analyte detection zone, wherein said control zone comprises bound a second tracer directed toward said second permeable conjugate pad unfixed ligand and forms a visible complex upon binding; and viii) an absorbent pad that is in contact with the distal end of said strip for absorbing liquid from said strip; b) applying the sample via said sample port to said test cassette; and c) detecting a visual indicator to establish a presence of the analyte.

A method utilizing lateral flow immunoassay (LIFA) for detecting of an analyte in a sample of saliva from a dog, wherein exposure to a virus selected from a group consisting of canine parvovirus (CPV) and canine distemper virus (CDV) is detected, comprising the steps of: a) providing a test cassette, wherein said test cassette comprises: i) a test cassette housing; ii) a sample port formed in said test housing that accesses a sample pad for receiving the body fluid sample; iii) a strip of material that allows lateral flow of liquid and is fitted within said cassette housing, wherein said strip of material is in contact with said sample pad; iv) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate; v) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate; vi) a first tracer affixed to said strip in an analyte detection zone that is downstream from said second permeable conjugate pad, wherein said tracer forms a detectable complex producing a visible mark upon encountering said third conjugate in said analyte detection zone; vii) a control zone downstream of said analyte detection zone, wherein said control zone comprises bound a second tracer directed toward said second permeable conjugate pad unfixed ligand and forms a visible complex upon binding; and viii) an absorbent pad that is in contact with the distal end of said strip for absorbing liquid from said strip; b) applying the sample via said sample port to said test cassette; and c) detecting a visual indicator to establish a presence of the analyte.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

### PATENT REFERENCES

4168146, Grubb, et al., September 1979.
4235601, Deutsch, et al., November 1980.
4366241, Tom, et al., December 1982.
4442204, Greenquist, et al., April 1984.
4632901, Valkirs, et al., December 1986.
4636479, Martin, et al., January 1987.
4703017, Campbell, et al., October 1987.
4775636, Moeremans, October 1988.
4376110, David, et al., March 1983.
4855240, Rosenstein, et al., August 1989.
4943522, Eisinger, et al., July 1990.
5073484, Swanson, December 1991.
WO 92/12428, Pawlak, et al., July 1992.
5415994, Imrich, et al., May 1995.
6656744, Pronvost, et al., December 2003.
6699722, Bauer, et al., March 2004.
20070087451, Kirkegarrd, April 2007.
20090246886, Buck, October 2009.
20110201131 A1, Badwan, et al., August 2011.

### ARTICLE REFERENCES

Adams, A., (1973). Concentration of Epstein-Barr virus from cell culture fluids with polyethylene glycol. J. Gen. Virol. 20:391-394.
Adler, B. and S. Faine (1983b). A Pomona serogroup-specific, agglutinating antigen in Leptospira, identified by monoclonal antibodies. Pathology 15:247-250.
Anderson, L., et al. (1986). Identification of epitopes on respiratory syncytial virus proteins by competitive binding immunoassay. J clin microbial. March: 475-480.
Appel, M. J., et al. (1979) Canine viral enteritis. I. Status report on corona- and parvo-like enteritides. Cornel vet. 69:123-133.
Batt, R., et al. (1991) relative IgA deficiency and small intestinal bacterial overgrowth in German shepherd dogs. Res vet sci. 50:106-111.
Bloom, M. E. (1997). Expression of Aleutian mink disease parvovirus capsid proteins in defined segments: localization of immunoreactive sites and neutralizing epitopes to specific regions. J. Virol. January; 71(1):705-714.
Bruning, A., et al. (1999). A rapid chromatographic strip test for the pen-side diagnosis of rinderpest virus. J. virol meth. 81:143-154.
Carmichael, L. E. (1980). Hemagglutination by canine parvovirus: serologic studies and diagnostic applications. Amer J Vet Res. May; 41(5):784-791.
Cytodiagnostics, Techical note #102, Adsorption of proteins to gold nanoparticles
Fooks, A., et al. (1993). Measles virus nucleocapsid protein expressed in insect cells assembles into nucleocapsi-like structures. J gen virol. 74:1439-1444.
Friedman, M. (1982) Radioimmunoassay for the Detection of Virus Specific Antibodies in Saliva. J immunol meth, 54:203-211.
German, A. J., et. al. (1998). Measurement of IgG, IgM, and IgA concentrations in canine serum, saliva, tears, and bile. Vet immunol immunopathol. July 8; 64(2):107-121.
Greene, C. E. and Levy, J. K. (2012). Immunoprophylaxis, Ch 100, in Greene's Infectious Diseases of the Dog and Cat, ed Greene, C, E., Elsevier, St. Louis, Mo., pp 1171-1176.
Goyal, A., et. al. (2009). Oral fluid, a substitute for serum to monitor measles IgG antibody? Indian J Med Microbiol. 27(4):351-353.
Heddle, R., and D. Rowley. (1975). Dog immunoglobulins I & II. Immunol. 29:185-208.
Hummer, K., et al. (1992). Baculovirus expression of the nucleoprotein gene of measles virus and utility of the recombinant protein in diagnostic enzyme immunoassays. J clin microbiol, November 30(11): 2874-2880.
Ismail, T., et al. (1994). Cloning and expression of the nucleocapsid gene of virulent Kabete O strain of rinderpest virus in baculovirus: use in differential diagnosis between vaccinated and infected animals. Virology. January; 198(1): 138-147.
Iwatsuki, K., et al. (1997). Molecular and phylogenetic analyses of the haemagglutinin (H) proteins of field isolates of canine distemper virus from naturally infected dogs. J gen virol. 78:373-380.
Kerr, M. A. (1990). The structure and function of human IgA. Biochem J 271:285-296.
Kikkawa, A., et. al. (2003). Salivary secretory IgA concentrations in beagle dogs. J. Vet Med. Sci. 65(6):689-693.
Kinder, A., et al. (1992). Salivary and serum antibodies in experimental canine taeniasis. Vet parasitol. 41:321-327.
Kohler, G., and C. Milstein. (1975). Continuous cultures of fused cells secreting antibody of predefined specificity. Nature 256:495-497.
Kremer, J. R., and Muller, C. P. (2005). Evaluation of commercial assay detecting specific immunoglobulin in oral fluid for determining measles immunity in vaccines. Clin Diagn. Lab Immunol. May; 12(5):668-670.
Martella, V., et. al. (2005). Surveillance activity for canine parvovirus in Italy. J Vet Med B Infect Dis Vet Public Health. September-October; 52(7-8):312-315.
Marulappa, S. Y. and S. Kapil. (2008). Simple tests for rapid detection of canine parvovirus antigen and canine parvovirus-specific antibodies. Clin Vacc Immunol. January; 16(1):127-131.
Mestecky, J., et al. (1987). Induction and molecular properties of secretory and serum IgA antibodies specific for environmental antigens. Annals of allergy; November, 59:54-59.
McCaw, D. L., et al. (1998). Serum distemper virus and parvovirus antibody titers among dogs brought to a veterinary hospital for revaccination. J. Am. Vet Med Assoc. July 1; 213(1):72-75.
Mochizuki, M. et. al. (1993). Antigenic and genomic variabilities among recently prevalent parvoviruses of canine and feline origin in Japan. Vet Microbiol. December; 38(1-2):1-10.
Nigatu, W., et. al. (1999). Detection of measles specific IgG in oral fluid using and FITC/anti-FITC IgG capture enzyme linked immunsorbent assay (GACELISA). J Virol Methods. December; 83(1-2):135-144.
Nokes, D. J., et al. (2001). Has oral fluid the potential to replace serum for the evaluation of population immunity levels? A study of measles, rubella, and hepatitis B in rural Ethiopia. Bull World Health Organ. 79(7):588-595.
Ogra, P. L., et al. (1968). Immunoglobulin response in serum and secretions after immunization with alive and inactivated polio vaccine and natural infection. N Engl J Med. 279:893-900.
Oh, J. S., et al. (2006). One-step immunochromatography assay kit for detecting antibodies to canine parvovirus. Clin vaccine immunol. April; 13(4):520-524.
Oliver, C. (2010). Preparation of colloidal gold. Methods mol boil. 588:363-367.
Pollock, R. V., and L. E. Carmichael (1982). Dog response to inactivated canine parvovirus and feline panleukopenia virus vaccines. Cornell Vet. January; 72(1):16-35.
Sozemen, M. et al. (1996). IgA, M, and G plasma secreting cells are scattered around the various salivary glands, but not evenly distributed among the various glands. Vet res; 27:285-294
Takahashi, A., et al. (2009). Immunochromatographic assay using gold nanoparticles for measuring salivary secretory IgA in dogs as a stress marker. Sci technol adv mater; 10:1-5.
Tomasi, T. B. And J. Bienenstock (1968). Secretory immunoglobulins. Adv Immunol. 9:1-96.
Trifthauser, C., D. W. Hayden, and E. H. Beutner (1970). Procedures for immunization of goats human with immunoglobulins and complement. Int arch allergy appl immunol. 38:315-319.
Von Messing, V., et al. (1999). Rapid and sensitive detection of immunoglobulin M (IgM) and IgG antibodies against canine distemper virus by a new recombinant nucleocapsid protein-based enzyme-linked immunsorbent assay. J. clin microbial. April, 37(4):1049-1056.
Waner, T., et al. (2003). Evaluation of a dot ELISA kit for measuring immunoglobulin M antibodies to canine parvovirus and distemper virus. Vet Rec. 152:588-591.
Waner, T., et al. (1996). Assessment of maternal antibody decay and response to canine parvovirus vaccination using a clinic-based enzyme-linked immunsorbent assay. J. Vet. Diagn. Investig. 8:427-432.
Yang, D. K. (2010). Serological survey for canine parvovirus type 2a (CPV-2a) in stray dogs in south Korea. J. bacterial and virology; 40(2): 77-81.
Zipperle, L., et al. (2010). Identification of key residues in virulent canine distemper virus hemagglutinin that control CD150/SLAM-binding activity. J virol, September 84(18):9618-9624.

1. A lateral flow immunoassay (LFIA) apparatus for detection of an analyte in a body fluid sample from an animal pet, utilizing a test cassette, comprised of:
a) a test cassette housing;
b) a sample port formed in said test cassette housing for accessing a sample pad;
c) said sample pad accessed via said sample port for receiving the body fluid sample, wherein said sample pad filters out test-interfering glycoproteins and interfering antibodies and wherein said sample pad contains a polyclonal antibody (Pab) selected from the group consisting of Pab anti-IgG and Pab anti-IgA;
d) a strip of material that allows lateral flow of liquid and is fitted within said cassette housing, wherein said strip of material is in contact with said sample pad, wherein said strip has an upstream proximal end and a downstream distal end;
e) a first permeable conjugate pad in contact with said strip, wherein said first permeable conjugate pad contains a species selected from the group consisting of unfixed ligand to which the analyte will bind and antibody proteins that compete with the analyte for a ligand, wherein said first pad species is coupled with biotin to produce a first conjugate;
f) a second permeable conjugate pad is downstream of said first permeable conjugate pad and in contact with said lateral flow strip, wherein said second permeable conjugate pad contains an unfixed-to-strip ligand that does not bind to the analyte and is avidin bound to a detectable label species thereby forming a second conjugate, wherein said first conjugate encounters and binds to said second conjugate to form a third conjugate;
g) a first tracer affixed to said strip in an analyte detection zone that is downstream from said second permeable conjugate pad, wherein said tracer forms a detectable complex producing a visible mark upon encountering said third conjugate in said analyte detection zone;
h) a control zone downstream of said analyte detection zone, wherein said control zone comprises a bound a second tracer directed toward said second permeable conjugate pad unfixed-to-strip ligand and forms a visible complex upon binding; and
i) an absorbent pad that is in contact with the distal end of said strip for absorbing liquid from said strip.

2. A lateral flow immunoassay (LFIA) apparatus for detection of an analyte in a sample of saliva from a dog, wherein exposure to a virus selected from a group consisting of canine parvovirus (CPV) and canine distemper virus (CDV) is detected, utilizing a test cassette, comprised of:
a) a test cassette housing;
b) a sample port formed in said test cassette housing for accessing a sample pad;
c) said sample pad accessed via said sample port for receiving the saliva sample, wherein said sample pad filters out test-interfering glycoproteins and interfering antibodies and wherein said sample pad contains a polyclonal antibody (Pab) selected from the group consisting of Pab anti-IgG and Pab anti-IgA;
d) a strip of material that allows lateral flow of liquid and is fitted within said cassette housing, wherein said strip of material is in contact with said sample pad, wherein said strip has an upstream proximal end and a downstream distal end;
e) a first permeable conjugate pad in contact with said strip, wherein said first permeable conjugate pad contains a species selected from the group consisting of unfixed ligand to which the analyte will bind and antibody proteins that compete with the analyte for a ligand, wherein said first pad species is coupled with biotin to produce a first conjugate;
f) a second permeable conjugate pad is downstream of said first permeable conjugate pad and in contact with said lateral flow strip, wherein said second permeable conjugate pad contains an unfixed-to-strip ligand that does not bind to the analyte and is avidin bound to a detectable label species thereby forming a second conjugate, wherein said first conjugate encounters and binds to said second conjugate to form a third conjugate;
g) a first tracer affixed to said strip in an analyte detection zone that is downstream from said second permeable conjugate pad, wherein said tracer forms a detectable complex producing a visible mark upon encountering said third conjugate in said analyte detection zone;
h) a control zone downstream of said analyte detection zone, wherein said control zone comprises a bound a second tracer directed toward said second permeable conjugate pad unfixed-to-strip ligand and forms a visible complex upon binding; and
i) an absorbent pad that is in contact with the distal end of said strip for absorbing liquid from said strip.

3. A lateral flow immunoassay (LIFA) apparatus for detection of an analyte in a sample of saliva from a dog according to claim 2, wherein the analyte is IgA and the detection is by a direct method, wherein: said sample pad contains Pab anti-IgG; said first conjugate pad contains CPV/VP2-biotin; and said second conjugate pad contains avidin-gnp.

4. The lateral flow immunoassay (LFIA) apparatus for detection of an analyte in a sample of saliva from a dog of claim 2, wherein the analyte is CPV-specific IgG and the detection is by a direct method, wherein: said sample pad contains polyclonal antibody (Pab) anti-IgA; said first conjugate pad contains canine parvovirus/VP2 antigen (CPV/VP2)-biotin; and said second conjugate pad contains avidin-gold nanoparticle (gnp).

5. A lateral flow immunoassay (LIFA) apparatus for detection of an analyte in a sample of saliva from a dog according to claim 2, wherein the analyte is IgA and the detection is by a direct method, wherein: said sample pad contains Pab anti-IgG; said first conjugate pad contains CDV N-protein-biotin; and said second conjugate pad contains avidin-gnp.

6. A lateral flow immunoassay (LIFA) apparatus for detection of an analyte in a sample of saliva from a dog according to claim 2, wherein the analyte is IgG and the detection is by a direct method, wherein: said sample pad contains Pab anti-IgA; said first conjugate pad contains CDV N-protein-biotin; and said second conjugate pad contains avidin-gnp.

7. A lateral flow immunoassay (LIFA) apparatus for detection of an analyte in a sample of saliva from a dog according to claim 2, wherein the analyte is IgA and the detection is by a competitive method, wherein: said sample pad contains Pab anti-IgG; said first conjugate pad contains IgA or IgG anti-CVP-biotin; and said second conjugate pad contains avidin-gnp.

8. A lateral flow immunoassay (LIFA) apparatus for detection of an analyte in a sample of saliva from a dog according to claim 2, wherein the analyte is IgG and the detection is by a competitive method, wherein: said sample pad contains Pab anti-IgA; said first conjugate pad contains IgG anti-CVP-biotin; and said second conjugate pad contains avidin-gnp.

9. A lateral flow immunoassay (LIFA) apparatus for detection of an analyte in a sample of saliva from a dog according to claim 2, wherein the analyte is IgA and the detection is by a competitive method, wherein: said sample pad contains Pab anti-IgG; said first conjugate pad contains IgA or IgG anti-CDV-biotin; and said second conjugate pad contains avidin-gnp.

10. A lateral flow immunoassay (LIFA) apparatus for detection of an analyte in a sample of saliva from a dog according to claim 2, wherein the analyte is IgG and the detection is by a competitive method, wherein: said sample pad contains Pab anti-IgA; said first conjugate pad contains IgG anti-CDV-biotin; and said second conjugate pad contains avidin-gnp.

11. A lateral flow immunoassay (LIFA) apparatus for detection of an analyte in a sample of saliva from a dog according to claim 2, wherein the analyte is IgA and the detection is by a double sandwich assay method, wherein: said sample pad contains Pab anti-IgG; said first conjugate pad contains Mab anti-IgA Fc-biotin; and said second conjugate pad contains avidin-gnp, wherein a bound ligand is CPV/VP2 prot.

12. A lateral flow immunoassay (LIFA) apparatus for detection of an analyte in a sample of saliva from a dog according to claim 2, wherein the analyte is IgG and the detection is by a double sandwich assay method, wherein: said sample pad contains Pab anti-IgA; said first conjugate pad contains Mab anti-IgG Fc-biotin; and said second conjugate pad contains avidin-gnp, wherein a bound ligand is CPV/VP2 prot.

13. A lateral flow immunoassay (LIFA) apparatus for detection of an analyte in a sample of saliva from a dog according to claim 2, wherein the analyte is IgA and the detection is by a double sandwich assay method, wherein: said sample pad contains Pab anti-IgG; said first conjugate pad contains Mab anti-IgA Fc-biotin; and said second conjugate pad contains avidin-gnp, wherein a bound ligand is CDV N-prot.

14. A lateral flow immunoassay (LIFA) apparatus for detection of an analyte in a sample of saliva from a dog according to claim 2, wherein the analyte is IgG and the detection is by a double sandwich assay method, wherein: said sample pad contains Pab anti-IgA; said first conjugate pad contains Mab anti-IgG Fc-biotin; and said second conjugate pad contains avidin-gnp, wherein a bound ligand is CDV N-prot.

15. A method utilizing lateral flow immunoassay (LIFA) for detecting an analyte in a body fluid sample from an animal pet, comprising the steps of:
a) providing a test cassette, wherein said test cassette comprises:
i) a test cassette housing;
ii) a sample port formed in said test housing that accesses a sample pad for receiving the body fluid sample;
iii) a strip of material that allows lateral flow of liquid and is fitted within said cassette housing, wherein said strip of material is in contact with said sample pad;
iv) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate;
v) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate;
vi) a first tracer affixed to said strip in an analyte detection zone that is downstream from said second permeable conjugate pad, wherein said tracer forms a detectable complex producing a visible mark upon encountering said third conjugate in said analyte detection zone;
vii) a control zone downstream of said analyte detection zone, wherein said control zone comprises a second bound tracer directed toward said second permeable conjugate pad unfixed ligand and forms a visible complex upon binding; and
viii) an absorbent pad that is in contact with the distal end of said strip for absorbing liquid from said strip;
b) applying the sample via said sample port to said test cassette; and
c) detecting a visual indicator to establish a presence of the analyte.

16. A method utilizing lateral flow immunoassay (LIFA) for detecting of an analyte in a sample of saliva from a dog, wherein exposure to a virus selected from a group consisting of canine parvovirus (CPV) and canine distemper virus (CDV) is detected, comprising the steps of:
a) providing a test cassette, wherein said test cassette comprises:
i) a test cassette housing;
ii) a sample port formed in said test housing that accesses a sample pad for receiving the body fluid sample;
iii) a strip of material that allows lateral flow of liquid and is fitted within said cassette housing, wherein said strip of material is in contact with said sample pad;
iv) a first permeable conjugate pad in contact with said strip, wherein said first permeable conjugate pad contains a species selected from a group consisting of unfixed ligand to which the analyte will bind and antibody proteins that compete with the analyte for a ligand, wherein said first pad species is coupled with biotin to produce a first conjugate;
v) a second permeable conjugate pad is downstream of said first permeable conjugate pad and in contact with said lateral flow strip, wherein said second permeable conjugate pad contains an unfixed ligand that does not bind to the analyte and is unbound avidin bound to a detectable label species thereby forming a second conjugate, wherein said first conjugate encounters and binds to said second conjugate to form a third conjugate;
vi) a first tracer affixed to said strip in an analyte detection zone that is downstream from said second permeable conjugate pad, wherein said tracer forms a detectable complex producing a visible mark upon encountering said third conjugate in said analyte detection zone;
vii) a control zone downstream of said analyte detection zone, wherein said control zone comprises bound a second tracer directed toward said second permeable conjugate pad unfixed ligand and forms a visible complex upon binding; and
viii) an absorbent pad that is in contact with the distal end of said strip for absorbing liquid from said strip;
b) applying the sample via said sample port to said test cassette; and
c) detecting a visual indicator to establish a presence of the analyte.

17. A method for detection of an analyte in a sample of saliva from a dog according to claim 16, wherein the analyte is IgA and the detection is by a direct method, wherein: said sample pad contains Pab anti-IgG; said first conjugate pad contains CPV/VP2-biotin; and said second conjugate pad contains avidin-gnp.

18. A method for detection of an analyte in a sample of saliva from a dog according to claim 16, wherein the analyte is IgG and the detection is by a direct method, wherein: said sample pad contains Pab anti-IgA; said first conjugate pad contains CPV/VP2-biotin; and said second conjugate pad contains avidin-gnp.

19. A method for detection of an analyte in a sample of saliva from a dog according to claim 16, wherein the analyte is IgA and the detection is by a direct method, wherein: said sample pad contains Pab anti-IgG; said first conjugate pad contains CDV N-protein-biotin; and said second conjugate pad contains avidin-gnp.

20. A method for detection of an analyte in a sample of saliva from a dog according to claim 16, wherein the analyte is IgG and the detection is by a direct method, wherein: said sample pad contains Pab anti-IgA; said first conjugate pad contains CDV N-protein-biotin; and said second conjugate pad contains avidin-gnp.

21. A method for detection of an analyte in a sample of saliva from a dog according to claim 16, wherein the analyte is IgA and the detection is by a competitive method, wherein: said sample pad contains Pab anti-IgG; said first conjugate pad contains IgA or IgG anti-CVP-biotin; and said second conjugate pad contains avidin-gnp.

22. A method for detection of an analyte in a sample of saliva from a dog according to claim 16, wherein the analyte is IgG and the detection is by a competitive method, wherein: said sample pad contains Pab anti-IgA; said first conjugate pad contains IgG anti-CVP-biotin; and said second conjugate pad contains avidin-gnp.

23. A method for detection of an analyte in a sample of saliva from a dog according to claim 16, wherein the analyte is IgA and the detection is by a competitive method, wherein: said sample pad contains Pab anti-IgG; said first conjugate pad contains IgA or IgG anti-CDV-biotin; and said second conjugate pad contains avidin-gnp.

24. A method for detection of an analyte in a sample of saliva from a dog according to claim 16, wherein the analyte is IgG and the detection is by a competitive method, wherein: said sample pad contains Pab anti-IgA; said first conjugate pad contains IgG anti-CDV-biotin; and said second conjugate pad contains avidin-gnp.

25. A method for detection of an analyte in a sample of saliva from a dog according to claim 16, wherein the analyte is IgA and the detection is by a double sandwich assay method, wherein: said sample pad contains Pab anti-IgG; said first conjugate pad contains Mab anti-IgA Fc-biotin; and said second conjugate pad contains avidin-gnp, wherein a bound ligand is CPV/VP2 prot.

26. A method for detection of an analyte in a sample of saliva from a dog according to claim 16, wherein the analyte is IgG and the detection is by a double sandwich assay method, wherein: said sample pad contains Pab anti-IgA; said first conjugate pad contains Mab anti-IgG Fc-biotin; and said second conjugate pad contains avidin-gnp, wherein a bound ligand is CPV/VP2 prot.

27. A method for detection of an analyte in a sample of saliva from a dog according to claim 16, wherein the analyte is IgA and the detection is by a double sandwich assay method, wherein: said sample pad contains Pab anti-IgG; said first conjugate pad contains Mab anti-IgA Fc-biotin; and said second conjugate pad contains avidin-gnp, wherein a bound ligand is CDV N-prot.

28. A method for detection of an analyte in a sample of saliva from a dog according to claim 16, wherein the analyte is IgG and the detection is by a double sandwich assay method, wherein: said sample pad contains Pab anti-IgA; said first conjugate pad contains Mab anti-IgG Fc-biotin; and said second conjugate pad contains avidin-gnp, wherein a bound ligand is CDV N-prot.
